(19) United States
(12) Patent Application Publication
(10) Pub. No.: US 2009/0287091 A1
(43) Pub. Date: Nov. 19, 2009

(54) Apparatus and method for generating high resolution image of human body using terahertz electromagnetic wave and endoscope using the same

(75) Inventors: Joo-Hiuk SON, Seoul (KR); Seung Jae OH, Seoul (KR); Seungjoo HAAM, Seoul (KR); Young-Min HUH, Seoul (KR); Jinyoung KANG, Seoul (KR); Inhee MAENG, Seoul (KR)

Correspondence Address: THE NATH LAW GROUP, 112 South West Street, Alexandria, VA, 22314, US

(73) Assignee: UNIVERSITY OF SEOUL FOUNDATION OF INDUSTRY ACADEMIC COOPERATION, Seoul (KR)

(21) Appl. No.: 12/385,044

(22) Filed: Mar. 30, 2009

(30) Foreign Application Priority Data
May 14, 2008 (KR) 10-2008-0044648
Feb. 19, 2009 (KR) 10-2009-0014015

(51) Int. Cl. A61B 1/06; A61B 6/00

(52) U.S. Cl. 600/473; 600/478; 600/182

(57) ABSTRACT

An apparatus and a method for generating a high resolution image of a human body using a terahertz electromagnetic wave and an endoscope using the same are disclosed. In accordance with the present invention, a third laser beam and a terahertz electromagnetic wave excited by a first laser beam are radiated on a portion of a human body having a contrast agent adhered thereto to generate a high resolution image based on the terahertz electromagnetic wave reflected from the portion of the human body and a second laser beam.

This application claims the benefit of Korean Patent Application No. 10-2008-0044648 filed on May 14, 2008 and Korean Patent Application No. 10-2009-0014015 filed on Feb. 19, 2009 which are hereby incorporated for reference.

### BACKGROUND OF THE INVENTION

1. Field of the Invention

The present invention relates to an apparatus and a method for generating a high resolution image of a human body using a terahertz electromagnetic wave and an endoscope using the same, and more particularly to an apparatus and a method for generating a high resolution image of a human body using a terahertz electromagnetic wave and an endoscope using the same wherein a third laser beam and a terahertz electromagnetic wave excited by a first laser beam are radiated on a portion of a human body having a contrast agent adhered thereto to generate a high resolution image based on the terahertz electromagnetic wave reflected from the portion of the human body and a second laser beam.

2. Description of the Related Art

An endoscope is a medical instrument for observing an organ without a surgery.

Generally, the endoscope may be classified into a rigid borescope wherein a lens attached to an end portion of a long and thin tube is employed, a flexible fiberscope that employs differential a glass fiber, and a capsule endoscope.

The endoscope provides an internal image of the organ. Experts such as doctors refer to the internal image of the organ provided by the endoscope to diagnose a tumor. Therefore, results of the diagnosis may differ according to expertise of the experts. Moreover, since the experts diagnose the tumor based on the internal image of the organ, the tumor may be diagnosed only after the tumor has progressed to a certain stage rather than in its early stage.

Therefore, the experts must use an x-ray machine, a computed tomography (CT) scanner or a magnetic resonance imaging (MRI) equipment such the medical instrument in order to diagnose a state of the organ accurately.

The x-ray machine is the medical instrument for taking a snapshot of the organs of the human body using an x-ray. The CT scanner is the medical instrument wherein a density of the human body having the x-ray passed therethrough is analyzed to generate images of the organs in the human body. The MRI equipment is the medical instrument wherein a magnetic material composing the human body is measured to reconstruct the human body in images using a computer.

Because the MRI equipment does not use an ionized radioactive ray such as the x-ray, the diagnosis using the MRI equipment is harmless to the human body. In addition, the MRI equipment provides coronal and sagittal views which cannot be provided by the CT scanner as well as a three dimensional (3D) images having superior contrast and resolution compared to the CT scanner. Moreover, the MRI equipment is capable of generating an image of the human body in an arbitrary angle. The MRI equipment is thus used for a neurological imaging, a musculoskeletal imaging and a cardiovascular imaging.

Due to the above-described advantages, the MRI equipment is often used to diagnose the human body.

However, a cost for the diagnosis using the MRI equipment is high. Moreover, a time for the imaging using the MRI equipment is very long. In addition, a space allocated for a patient within the MRI equipment is very small such that only the patient can enter the space. Therefore, the MRI equipment cannot be used on the patient with a claustrophobia.

Particularly, the resolution of the MRI equipment is insufficient to be used to identify an incipient tumor.

### SUMMARY OF THE INVENTION

It is an object of the present invention to provide an apparatus and a method for generating a high resolution image of a human body using a terahertz electromagnetic wave and an endoscope using the same wherein a third laser beam and a terahertz electromagnetic wave excited by a first laser beam are radiated on a portion of a human body having a contrast agent adhered thereto to generate a high resolution image based on the terahertz electromagnetic wave reflected from the portion of the human body and a second laser beam.

It is another object of the present invention to provide an apparatus and a method for generating a high resolution image of a human body using a terahertz electromagnetic wave and an endoscope using the same wherein a third laser beam varying according to time and a terahertz electromagnetic wave excited by a first laser beam are radiated on a portion of a human body having a contrast agent adhered thereto to generate an image via a difference in an electrical signal generated from the terahertz electromagnetic wave reflected from the portion of the human body according to the varying in the third laser beam and a second laser beam thereby, improving a sensitivity for the image of the portion of the human body.

In accordance with a first aspect of the present invention, there is provided an apparatus for generating a high resolution image of a human body, the apparatus comprising: an electromagnetic wave generator for radiating a terahertz electromagnetic wave excited by a first laser beam on a portion of the human body having a contrast agent adhered thereto; a laser beam laser beam transmitter for radiating a third laser beam on the portion of the human body; a detector for generating an electric signal for an imaging based on the terahertz electromagnetic wave reflected from the portion of the human body having the third laser beam radiated thereon and a second laser beam; and an image generator for generating the high resolution image based on the electric signal received from the detector.

The apparatus in accordance with the present invention may further comprise: a first laser beam generator for generating the first laser beam; a first laser beam propagator for propagating the first laser beam generated by the first laser beam generator to the electromagnetic wave generator; a third laser beam generator for generating the third laser beam; a third laser beam propagator for propagating the third laser beam generated by the third laser beam generator to the laser beam laser beam transmitter; a second laser beam generator for generating the second laser beam; and a second laser beam propagator for propagating the second laser beam generated by the second laser beam generator to the detector.

Preferably, each of the first laser beam propagator, the third laser beam propagator and the second laser beam propagator includes an optical fiber.

The apparatus in accordance with the present invention may further comprise: a first collimation lens for focusing the first laser beam, the first collimation lens being interposed between the electromagnetic wave generator and the first laser beam propagator; a third collimation lens for focusing the third laser beam, the third collimation lens being interposed between the laser beam laser beam transmitter and the third laser beam propagator; and a second collimation lens for focusing the second laser beam, the second collimation lens being interposed between the detector and the second laser beam propagator.

The apparatus in accordance with the present invention may further comprise a first silicon lens for collimating the terahertz electromagnetic wave radiated by the electromagnetic wave generator.

The apparatus in accordance with the present invention may further comprise a second silicon lens for collimating the terahertz electromagnetic wave reflected from the portion of the human body.

The apparatus in accordance with the present invention may further comprise: a first cable for providing a power to the electromagnetic wave generator from a power supply; and a second cable for transmitting the electric signal generated by the detector to the image generator.

Preferably, the terahertz electromagnetic wave is excited by the first laser beam via one of a photoconductive antenna and an optical rectification.

Preferably, the detector generates the electric signal using one of a photoconductive sampling and an electro-optic sampling.

Preferably, the contrast agent includes one of a metal nanoparticle and a metal nanocluster including the metal nanoparticle.

Preferably, the metal nanoparticle includes at least one of shapes of a rod, an oval and a sphere.

Preferably, the metal nanoparticle includes at least one of a Pt, a Pd, an Ag, a Cu and an Au.

Preferably; the third laser beam includes one of an infrared laser beam and a visible ray laser beam.

Preferably, the image generator generates the high resolution image based on a difference in the electric signal varying according to a variation of the third laser beam.

Preferably, the third laser beam includes a square wave laser beam.

In accordance with a second aspect of the present invention, there is provided a high resolution endoscope comprising: a electromagnetic wave generator for radiating an terahertz electromagnetic wave excited by a first laser beam on a portion of a human body having a contrast agent adhered thereto; a laser beam laser beam transmitter for radiating a third laser beam on the portion of the human body; a detector for generating an electric signal for a imaging based on the terahertz electromagnetic wave reflected from the portion of the human body having the third laser beam radiated thereon and a second laser beam; an image generator for generating a high resolution image based on the electric signal received from the detector; a first laser beam propagator, a third laser beam propagator and a second laser beam propagator for propagating the first laser beam, the third laser beam and the second laser beam to the electromagnetic wave generator, the laser beam transmitter and the detector, respectively; a flexible tube; and a dome-shaped header attached to an end portion of the flexible tube, the dome-shaped header being inserted into the human body with the flexible tube; and wherein the dome-shaped header houses in the electromagnetic wave generator, the laser beam transmitter and the detector, and wherein the flexible tube houses in the first laser beam propagator, the third laser beam propagator and the second laser beam propagator.

The endoscope in accordance with the present invention may further comprise: a first laser beam generator for generating the first laser beam; a third laser beam generator for generating the third laser beam; and a second laser beam generator for generating the second laser beam.

The endoscope in accordance with the present invention may further comprise: a first collimation lens for focusing the first laser beam, the first collimation lens being interposed between the electromagnetic wave generator and the first laser beam propagator; a third collimation lens for focusing the third laser beam, the third collimation lens being interposed between the laser beam transmitter and the third laser beam propagator; and a second collimation lens for focusing the second laser beam, the second collimation lens being interposed between the detector and the second laser beam propagator.

The endoscope in accordance with the present invention may further comprise a first silicon lens for collimating the terahertz electromagnetic wave radiated by electromagnetic wave generator.

Preferably, the first silicon lens is disposed on a surface of or inside the dome-shaped header.

The endoscope in accordance with the present invention may further comprise a second silicon lens for collimating the terahertz electromagnetic wave reflected from the portion of the human body.

Preferably, the second silicon lens is disposed for one of an inside of the dome-shaped header.

The endoscope in accordance with the present invention may further comprise: a first cable for providing a power from a power supply to the electromagnetic wave generator; and a second cable for transmitting the electric signal generated by the detector to the image generator.

Preferably, the first cable and the second cable are disposed in the flexible tube.

Preferably, the terahertz electromagnetic wave is excited by the first laser beam via one of a photoconductive antenna and an optical rectification.

Preferably, the detector generates the electric signal using one of a photoconductive sampling and an electro-optic sampling.

Preferably, each of the first laser beam propagator, the third laser beam propagator and second laser beam propagator includes an optical fiber.

The endoscope in accordance with the present invention may further comprise a visible image generator for feeding a video of the portion of the human body, the visible image generator being housed in the dome-shaped header.

Preferably, the contrast agent includes one of a metal nanoparticle and a metal nanocluster including the metal nanoparticle.

Preferably, the metal nanoparticle includes at least one of shapes of a rod, an oval and a sphere.

Preferably, the metal nanoparticle includes at least one of a Pt, a Pd, an Ag, a Cu and an Au.

Preferably, the third laser beam includes one of an infrared laser beam and a visible ray laser beam.

Preferably, the image generator generates the high resolution image based on a difference in the electric signal varying according to a variation of the third laser beam.

Preferably, the third laser beam includes a square wave laser beam.

In accordance with a third aspect of the present invention, there is provided a method for generating a high resolution image of a human body, the method comprising: (a) coating a contrast agent on a portion of the human body; (b) radiating a terahertz electromagnetic wave excited by a first laser beam on the portion of the human body having the contrast agent adhered thereto; (c) radiating a third laser beam on the portion of the human body; (d) generating an electric signal for an imaging based on the terahertz electromagnetic wave reflected from the portion of the human body having the third laser beam radiated thereon and a second laser beam; and (e) generating an image from the electric signal.

Preferably, the step (c) comprises radiating the third laser beam varying according to time, and wherein the step (d) comprises generating the electric signal varying according to the third laser beam.

Preferably, the step (e) comprises generating the image based on a difference in electric signal varying according to the third laser beam.

The method in accordance with the present invention may further comprise (f) displaying the image on a display apparatus.

### BRIEF DESCRIPTION OF THE DRAWINGS

FIG. 1 is a diagram illustrating a spectrum of an electromagnetic wave including a frequency range of a terahertz electromagnetic wave.

FIG. 2 is a block diagram illustrating an apparatus for generating a high resolution image of a human body using a terahertz electromagnetic wave in accordance with the present invention.

FIG. 3 is a graph illustrating a periodic square wave laser beam in accordance with the present invention.

FIG. 4 is a graph depicting an amount of a terahertz electromagnetic wave reflected from a portion of a human body having a contrast agent adhered thereto according to a variation of a third laser beam in accordance with the present invention.

FIG. 5 illustrates electrical signals and images corresponding to a portion of a human body with and without a contrast agent in accordance with the present invention.

FIG. 6 illustrates images generated based on a terahertz electromagnetic wave reflected from a portion of a human body with and without a contrast agent in accordance with the present invention.

FIG. 7 is a flow diagram illustrating a method for generating a high resolution image of a human body using a terahertz electromagnetic wave in accordance with an embodiment of the present invention.

FIG. 8 is a flow diagram illustrating a method for generating a high resolution image of a human body using a terahertz electromagnetic wave in accordance with another embodiment of the present invention.

FIG. 9 is a block diagram illustrating a high resolution endoscope using a terahertz electromagnetic wave in accordance with the present invention.

FIG. 10 is a schematic diagram illustrating a high resolution endoscope using a terahertz electromagnetic wave in accordance with an embodiment of the present invention.

### DETAILED DESCRIPTION OF THE INVENTION

An apparatus and a method for generating a high resolution image of a human body using a terahertz electromagnetic wave and an endoscope using the same in accordance with the present invention will now be described in detail with reference to the accompanied drawings.

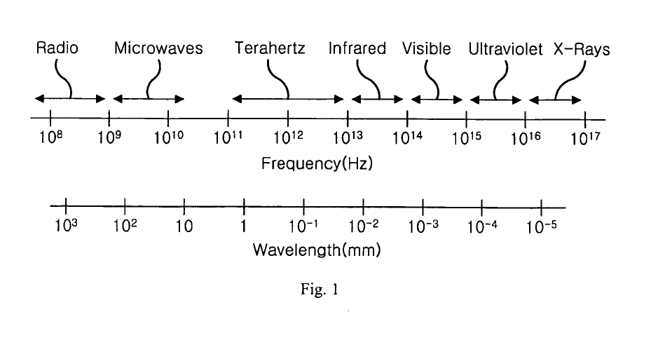

FIG. 1 is a diagram illustrating a spectrum of an electromagnetic wave including a frequency range of a terahertz electromagnetic wave.

As shown in FIG. 1, a band of the terahertz electromagnetic wave is between that of a microwave which is an electromagnetic wave and that of a far infrared laser beam which is a light wave in the spectrum. Therefore, the terahertz electromagnetic wave has a characteristic of both the electromagnetic wave and the light wave. That is, the terahertz electromagnetic wave, similar to a visible ray, has a characteristic of penetrating an object and traveling straight.

Generally, a frequency of the terahertz electromagnetic wave ranges from 0.1 terahertz (0.1×10¹² Hz) to 10 terahertz (10×10¹² Hz), and the terahertz electromagnetic wave oscillates at least 100 billion times per second.

The terahertz electromagnetic wave has a characteristic of penetrating a material which cannot be penetrated by the visible light wave. Moreover, the terahertz electromagnetic wave is generated by a combination of a laser beam having a pulse width of few femtoseconds and an optically conductive material having a carrier lifetime of less than few hundred femtoseconds.

The terahertz electromagnetic wave is very sensitive to a moisture. For instance, when a frequency of the terahertz electromagnetic wave is 1 terahertz, a rate of the terahertz electromagnetic wave absorbed by the moisture is very high about 230 cm⁻¹ such that the terahertz electromagnetic wave barely penetrates the object containing the moisture.

Particularly, since the moisture in a tumor such as a cancer increases as the tumor grows, a diagnosis of the tumor in the human body may be facilitated by utilizing the characteristic that the terahertz electromagnetic wave is sensitive to the moisture.

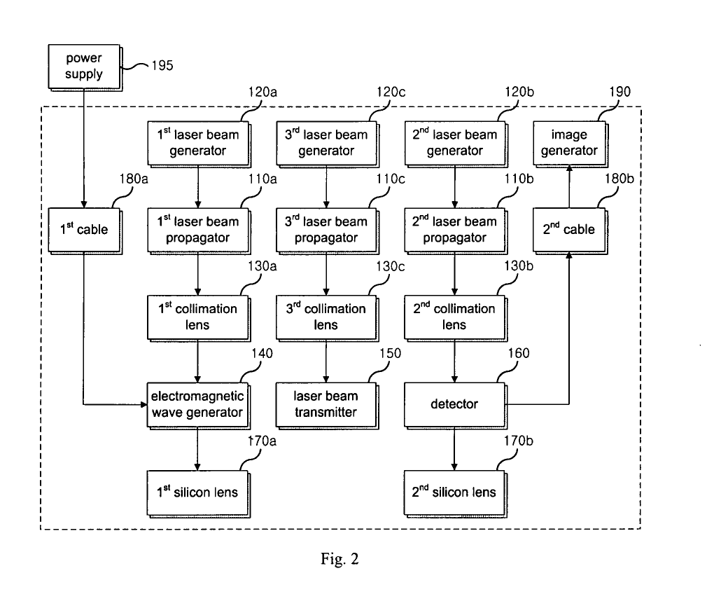

FIG. 2 is a block diagram illustrating an apparatus for generating a high resolution image of a human body using a terahertz electromagnetic wave in accordance with the present invention.

Referring FIG. 2, the apparatus for generating the high resolution image of the human body using the terahertz electromagnetic wave in accordance with the present invention comprises an electromagnetic wave generator 140, a laser beam transmitter 150, a detector 160 and an image generator 190.

In addition, the apparatus may further comprise a first laser beam generator 120a, a first laser beam propagator 110a, a third laser beam generator 120c, a third laser beam propagator 110c, a second laser beam generator 120b, a second laser beam propagator 110b, a first collimation lens 130a, a third collimation lens 130c, a second collimation lens 130b, a first silicon lens 170a, a second silicon lens 170b, a first cable 180a and a second cable 180b.

The first laser beam generator 120a generates a first laser beam. The first laser beam generated by the first laser beam generator 120a is propagated to the electromagnetic wave generator 140 via the first laser beam propagator 110a.

The third laser beam generator 120c generates a third laser beam. The third laser beam generated by the third laser beam generator 120c is propagated to the laser beam transmitter 150 via the third laser beam propagator 110c. Preferably, the third laser beam may include a laser beam that is constant and continuous or a laser beam varying according to time.

The second laser beam generator 120b generates a second laser beam. The second laser beam generated by the second laser beam generator 120b is propagated to the detector 160 via the second laser beam propagator 110b.

Each of the first laser beam propagator 110a, the third laser beam propagator 110c and the second laser beam propagator 110b may include an optical fiber. The first laser beam, the third laser beam and the second laser beam incident upon the first laser beam propagator 110a, the third laser beam propagator 110c and the second laser beam propagator 110b, respectively, are subjected to a total reflection and are propagated to the electromagnetic wave generator 140, the laser beam transmitter 150 and the detector 160, respectively.

The first collimation lens 130a is interposed between the first laser beam propagator 110a and the electromagnetic wave generator 140, and the first laser beam incident upon the first laser beam propagator 110a is focused by the first collimation lens 130a and propagated to the electromagnetic wave generator 140.

The third collimation lens 130c is interposed between the third laser beam propagator 110c and the laser beam transmitter 150, and the third laser beam incident upon the third laser beam propagator 110c is focused by the third collimation lens 130c and propagated to the laser beam transmitter 150.

The second collimation lens 130b is interposed between the second laser beam propagator 110b and the detector 160, and the second laser beam incident upon the second laser beam propagator 110b is focused by the second collimation lens 130b and propagated to the detector 160.

In one embodiment, each of the first collimation lens 130a, the third collimation lens 130c and the second collimation lens 130b may include an optical lens.

The laser beam transmitter 150 radiates the third laser beam on a portion of the human body having a contrast agent adhered thereto. It is preferable that the third laser beam includes one of an infrared laser beam and a visible ray laser beam.

An imaging using a contrast agent is possible because the tumor containing the excessive moisture absorbs most of the terahertz electromagnetic wave radiated by the electromagnetic wave generator 140. Moreover, the contrast agent tends to adhere to the tumor containing the excessive moisture when injected into the human body. Therefore, the contrast agent may increase the contrast for the tumor during the imaging.

In accordance with the present invention, the third laser beam is radiated on the portion of the human body having the contrast agent adhered thereto in order to further increase the contrast agent thereof.

That is, when the third laser beam is radiated on the portion of the human body having the contrast agent adhered thereto, a temperature of the moisture contained in the tumor, i.e. a temperature of the portion of the human body having the contrast agent adhered thereto rises due to a surface plasmon polariton phenomenon. Therefore, the tumor may easily be located owing to an increase of the contrast by the terahertz electromagnetic wave. In addition, a cancer may be killed due to the rise of the temperature.

Preferably, the contrast agent may include one of a metal nanoparticle and a metal nanocluster including the metal nanoparticle.

The metal nanoparticle may include at least one of a Pt (Platinum), a Pd (Palladium), an Ag (Silver), a Cu (Copper) and an Au (Gold). Moreover, it is preferable that the metal nanoparticle may have at least one of shapes of a rod, an oval and a sphere.

The metal nanocluster may be manufactured via an emulsion method using the metal nanoparticle.

The electromagnetic wave generator 140 radiates the terahertz electromagnetic wave excited by the first laser beam on the portion of the human body having the contrast agent adhered thereto.

The contrast agent maximizes a reaction of the terahertz electromagnetic wave radiated on the portion of the human body, thereby increasing a resolution of the imaging.

The terahertz electromagnetic wave may be excited by the first laser beam via one of a photoconductive antenna and an optical rectification.

The optical rectification utilizes a time-dependent polarization phenomenon, which is a non linear optical property, occurring when a strong light from a light signal source is received.

Specifically, a polarized microwave laser pulse is focused using a lens, and the focused microwave laser pulse is incident upon a photoconductive medium. A time-dependent electric field is formed in the photoconductive medium due to a polarization phenomenon generated by the incident microwave laser pulse. The time-dependent electric field lasts for an extremely short time. Accordingly, electrons in the photoconductive medium move in an accelerated manner to generate the terahertz electromagnetic wave.

The first silicon lens 170a collimates the terahertz electromagnetic wave radiated by the electromagnetic wave generator 140.

The second silicon lens 170b collimates the terahertz electromagnetic wave reflected from the portion of the human body.

Specifically, the first silicon lens 170a may collimate the excited terahertz electromagnetic wave radiated by the electromagnetic wave generator 140 to be locally radiated on the portion of the human body. In addition, the second silicon lens 170b may collimate the terahertz electromagnetic wave reflected from the portion of the human body to be propagated to the detector 160.

The detector 160 generates an electrical signal for the imaging based on the reflected terahertz electromagnetic wave and the second laser beam.

The detector 160 generates the electrical signal using one of a photoconductivity sampling and an electro-optic sampling.

The electro-optic sampling uses an electro-optic effect of an electro optical crystal.

An eletro-optic effect refers to a phenomenon wherein a refractive index changes with respect to an optical axis of an electro-optic medium by applying the electro-optic medium to an electric field. The electrical signal is generated by measuring a difference in the refractive index that appears when the terahertz microwave reaches and does not reach the electro-optic medium using Pockels effect as a change in an intensity of the microwave laser pulse.

The image generator 190 generates the high resolution image based on the electrical signal received from the detector 160. The high resolution image generated by the image generator 190 is displayed on a display apparatus (not shown).

As described above, the laser beam transmitter 150 may radiate the third laser beam that is constant and continuous or the third laser beam varying according to time.

A process of generating the high resolution image generated by the image generator 190 is described in more detail below.

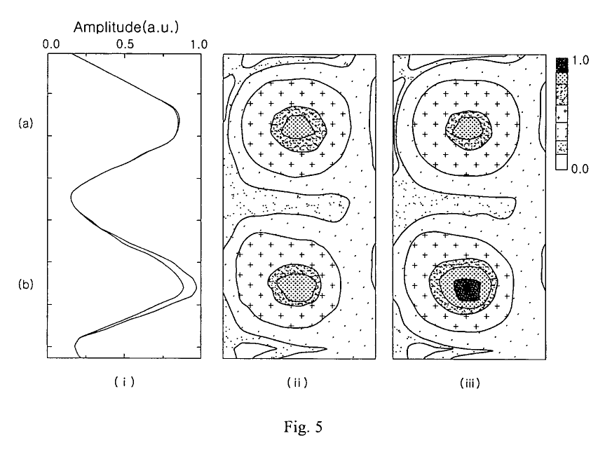

In accordance with an example wherein the third laser beam is constant and continuous, the image generator 190 generates the high resolution image from the electrical signal that is generated based on the terahertz electromagnetic wave reflected from the human body. That is, the image generator 190 generates an image as shown in (b)(iii) of FIG. 5 from the electrical signal generated by the detector 160. The generated image is displayed via the display apparatus.

Another example wherein the third laser beam varies according to time is described below in detail.

FIG. 4 is a graph exemplifying an amount of the terahertz electromagnetic wave reflected from the portion of the human body having the contrast agent adhered thereto according to a variation of the third laser beam in accordance with the present invention.

Referring FIG. 4, when the contrast agent of 3.2 mg/ml is used, and an intensity of the third laser beam is 2 W/cm², a reflectance of the terahertz electromagnetic wave reflected from the portion of the human body is about 5%, which is substantially maintained regardless of time. On the other hand, when the intensity of the third laser beam is 10 W/cm², the reflectance of the terahertz electromagnetic wave reflected from the portion of the human body is about 12.5%, which increases with time.

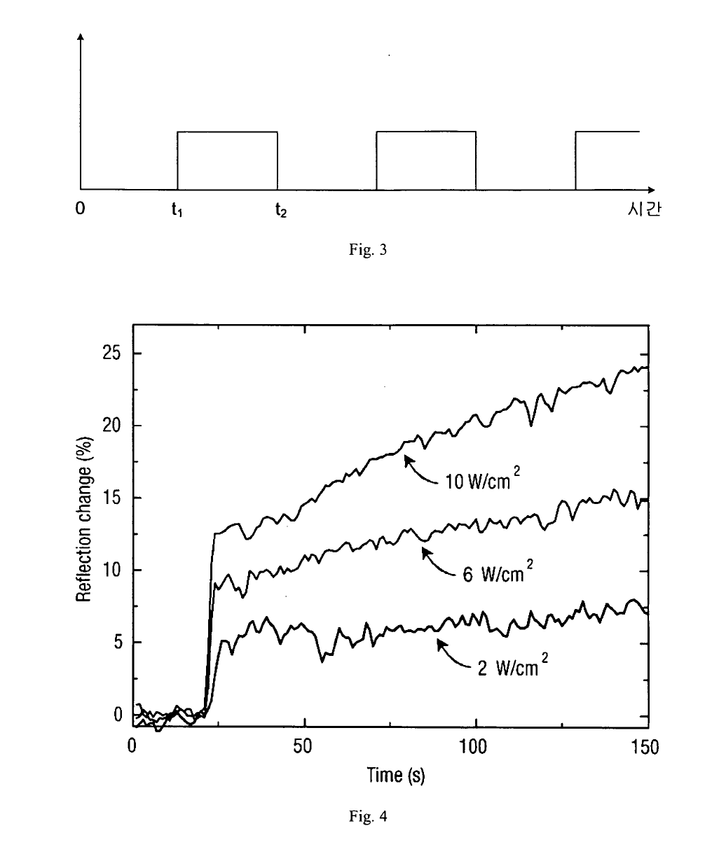

Therefore, when the third laser beam varying according to time, for instance, a periodic square wave laser beam shown in FIG. 3 having a level varying with time is radiated on the portion of the human body, the amount of the terahertz electromagnetic wave reflected from the portion of the human body varies according to the level. That is, an amplitude of the terahertz electromagnetic wave reflected from the portion of the human body for a time period [0˜t₁] of FIG. 3 during which the third laser beam is not radiated differ from that of a time period [t₁˜t₂] during which the third laser beam is radiated. The image generator 190 generates the image from the electrical signal generated by the detector 160 according to the amplitude of the terahertz electromagnetic wave reflected from the portion of the human body.

FIG. 5(a) is a drawing illustrating electrical signals obtained from the terahertz electromagnetic wave reflected from the portion of the human body without the contrast agent and images obtained from interpreting the electrical signal, and FIG. 5(b) is a drawing illustrating electrical signals obtained from the terahertz electromagnetic wave reflected from the portion of the human body with the contrast agent.

Particularly, (a)(i) of FIG. 5 is a graph illustrating the electrical signal generated based on the terahertz electromagnetic wave reflected from the portion of the human body without the contrast agent when only the terahertz electromagnetic wave without the third laser beam is radiated and both the terahertz electromagnetic wave and the third laser beam are radiated, i.e., the electrical signal generated during time periods [0˜t₁] and [t₁˜t₂], respectively. (a)(ii) and (a)(iii) of FIG. 5 depict images generated by interpreting the electrical signals of (a)(i) of FIG. 5.

Moreover, (b)(i) of FIG. 5 is a graph illustrating the electrical signal generated based on the terahertz electromagnetic wave reflected from the portion of the human body with the contrast agent when only the terahertz electromagnetic wave without the third laser beam is radiated and both the terahertz electromagnetic wave and the third laser beam are radiated, i.e., the electrical signal generated during time periods [0˜t₁] and [t₁˜t₂], respectively. (b)(ii) and (b)(iii) of FIG. 5 depict images generated by interpreting the electrical signals of (b)(i) of FIG. 5.

Referring (a) of FIG. 5, for the portion of the human body without the contrast agent (for instance, the portion of the human body without the tumor), there is no substantial difference between the amplitudes of the terahertz electromagnetic wave reflected during the time period [0˜t₁] and the time period [t₁˜t₂]. Therefore, there is no substantial difference between the electrical signals generated based on the terahertz electromagnetic wave reflected from the portion of the human body without the contrast agent.

On the other hand, referring (b) of FIG. 5, for the portion of the human body with the contrast agent (for instance, the portion of the human body with the tumor), there is substantial difference between the amplitudes of the terahertz electromagnetic wave reflected during the time period [0˜t₁] and the time period [t₁˜t₂]. Therefore, there is substantial difference between the electrical signals generated based on the terahertz electromagnetic wave reflected from the portion of the human body with the contrast agent.

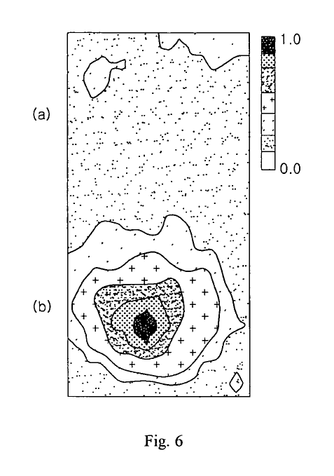

According to the characteristics described above, the difference between the electrical signal obtained during the time period [0˜t₁] and the electrical signal obtained during the time period [t₁˜t₂] is nearly zero, for the portion of the human body without the contrast agent, while the difference between the electrical signal obtained during the time period [0˜t₁] and the electrical signal obtained during the time period [t₁˜t₂] is not zero, for the portion of the human body with the contrast agent. When the image generator 190 generates the image based on the difference, the image shown in (a) of FIG. 6 is obtained for the portion of the human body without the tumor, and the image shown in (b) of FIG. 6 is obtained for the portion of the human body with the tumor.

Therefore, when the apparatus for generating the high resolution image of the human body using the terahertz electromagnetic wave in accordance with present invention is used, the apparatus accurately images the portion of the human body with the tumor and the portion of the human body without the tumor, thereby improving a sensitivity of detecting the tumor.

The first cable 180a provides a power from a power supply 195 to the electromagnetic wave generator 140.

The second cable 180b transmits the electrical signal generated by the detector 160 to the image generator 190.

For instance, each of the first cable 180a and the second cable 180b may include a copper cable.

A method for generating an image using the apparatus for generating a high resolution image of a human body using the terahertz electromagnetic wave in accordance with present invention is described in more detail below.

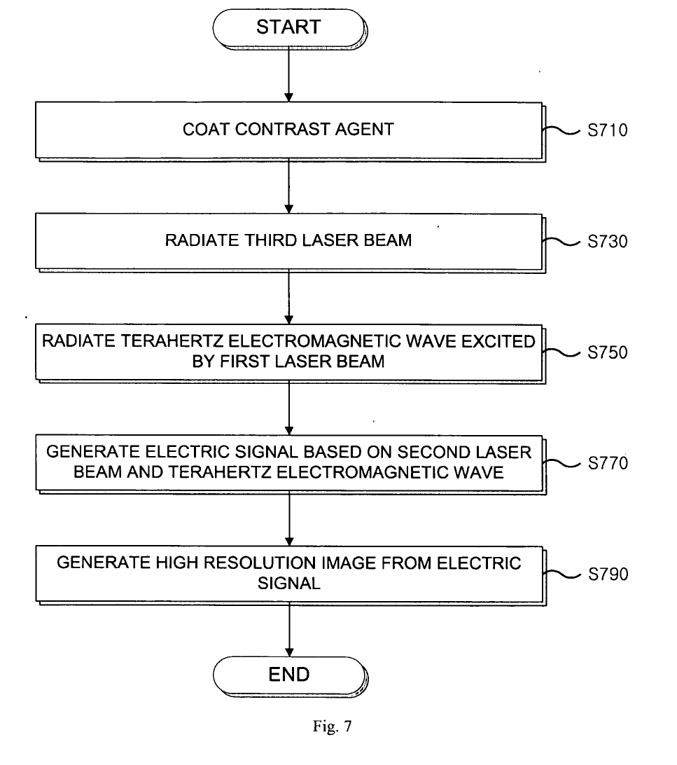

FIG. 7 is a flow diagram illustrating a method for generating a high resolution image of a human body using a terahertz electromagnetic wave in accordance with an embodiment of the present invention.

Referring FIG. 7, a contrast agent is coated on a portion of the human body (S710). Specifically, the contrast agent may include a metal nanoparticle or a metal nanocluster including the metal nanoparticle. The contrast agent tends to adhere to a tumor containing an excessive moisture such a cancer when injected into the human body.

Thereafter, a third laser beam is radiated on the portion of the human body (S730). Preferably, the third laser beam may include a laser beam that is constant and continuous. When the third laser beam is radiated on the portion of the human body having the contrast agent adhered thereto, a temperature of the portion of the human body having the contrast agent adhered thereto rises due to a surface plasmon polariton phenomenon.

Thereafter, the terahertz electromagnetic wave excited by a first laser beam is radiated on the portion of the human body (S750). When the temperature of the portion of the human body rises due to the surface plasmon polariton phenomenon, a contrast in an image of the portion of the human body increases, thereby allowing the high resolution imaging of the portion of the human body.

Thereafter, an electrical signal is generated based on a second laser beam and the terahertz electromagnetic wave reflected by the portion of the human body having the third laser beam radiated thereon (S770).

Thereafter, the high resolution image is generated from the electrical signal generated in the step S770 (S790). The image generator receives the electrical signal from the detector to generate the high resolution image. The high resolution image is displayed on a display apparatus.

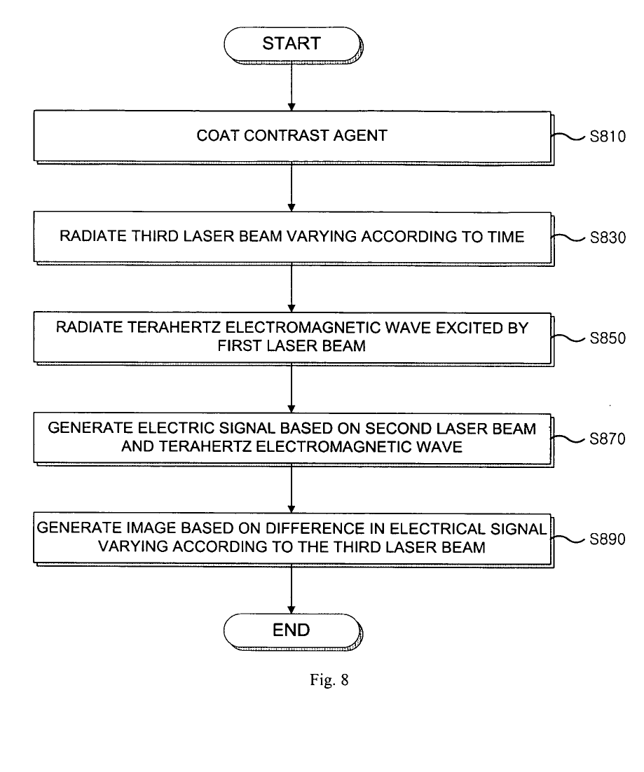

FIG. 8 is a flow diagram illustrating a method for generating a high resolution image of a human body using a terahertz electromagnetic wave in accordance with another embodiment of the present invention.

Referring FIG. 8, a contrast agent is coated on a portion of the human body (S810). Specifically, the contrast agent may include a metal nanoparticle or a metal nanocluster including the metal nanoparticle. The contrast agent tends to adhere to a tumor containing an excessive moisture such a cancer when injected into the human body.

Thereafter, a third laser beam is radiated on the portion of the human body (S830). Preferably, the third laser beam may include a laser beam varying according to time. When the third laser beam is radiated on the portion of the human body having the contrast agent adhered thereto, a temperature of the portion of the human body having the contrast agent adhered thereto rises due to a surface plasmon polariton phenomenon.

Thereafter, the terahertz electromagnetic wave excited by a first laser beam is radiated on the portion of the human body (S850). When the temperature of the portion of the human body rises due to the surface plasmon polariton phenomenon, a contrast in an image of the portion of the human body increases, thereby allowing the high resolution imaging of the portion of the human body.

Thereafter, an electrical signal is generated based on a second laser beam and the terahertz electromagnetic wave reflected by the portion of the human body having the third laser beam radiated thereon (S870).

Thereafter, the high resolution image is generated from the electrical signal generated in the step S870 (S890). An image generator receives the electrical signal generated based on the third laser beam, for instance a periodic square wave laser beam having a level varying with time, and generates the high resolution image based on a difference in the electrical signal. A process of generating the high resolution image generated by the image generator is described in FIGS. 5 and 6. Therefore, a detailed description thereof is omitted. The high resolution image is displayed on a display apparatus.

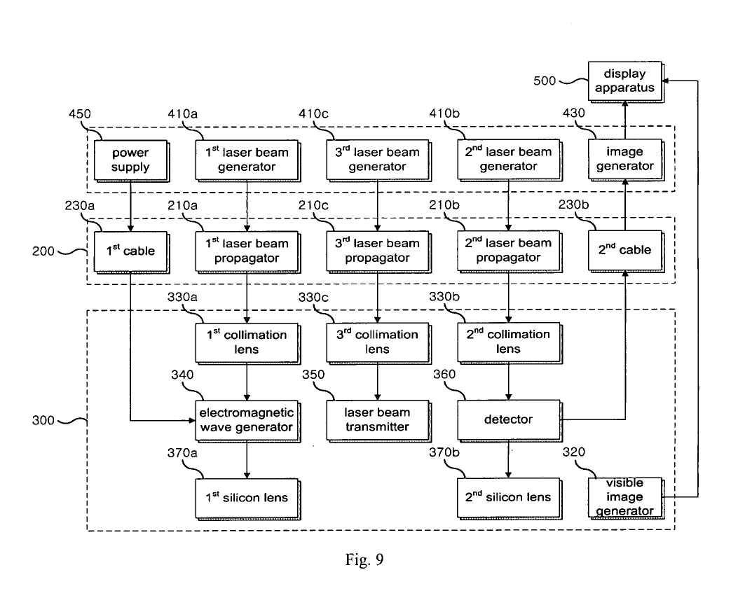

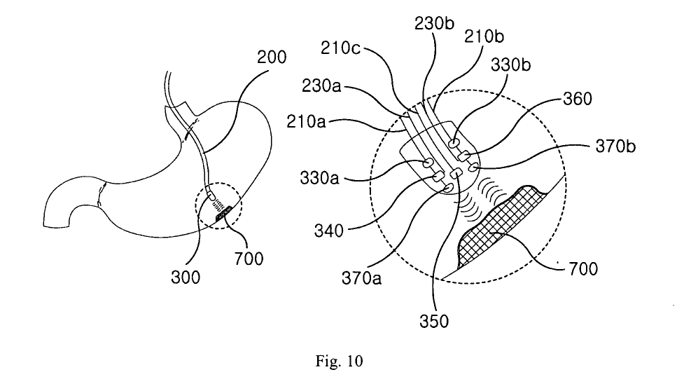

FIG. 9 is a block diagram illustrating a high resolution endoscope using a terahertz electromagnetic wave in accordance with the present invention, and FIG. 10 is a schematic diagram illustrating a high resolution endoscope using a terahertz electromagnetic wave in accordance with an embodiment of the present invention.

Referring FIGS. 9 and 10, a high resolution endoscope in accordance with the present invention comprises a flexible tube 200 and a dome-shaped header 300.

In addition, the endoscope may further comprise a first laser beam propagator 210a, a third laser beam propagator 210c and a second laser beam propagator 210b, which are housed in the flexible tube 200, and an electromagnetic wave generator 340, a laser beam transmitter 350 and a detector 360, which are housed in the dome-shaped header 300.

Moreover, the endoscope may further comprise a first laser beam generator 410a, a third laser beam generator 410c, a second laser beam generator 410b, a first collimation lens 330a, a third collimation lens 330c, a second collimation lens 330b, a first silicon lens 370a, a second silicon lens 370b, a first cable 230a, a second cable 230b, a visible image generator 320 and an image generator 430.

The flexible tube 200 is inserted into the human body along with the dome-shaped header 300 attached to an end portion thereof.

Preferably, the flexible tube 200 is composed of a thin and flexible material in order to facilitate a movement thereof inside the human body.

The flexible tube 200 houses the first laser beam propagator 210a, the third laser beam propagator 210c, the second laser beam propagator 210b, the first cable 230a and the second cable 230b.

The first laser beam propagator 210a, the third laser beam propagator 210c and the second laser beam propagator 210b propagate a first laser beam generated by the first laser beam generator 410a, a third laser beam generated by the third laser beam generator 410c and a second laser beam generated by the second laser beam generator 410b to the electromagnetic wave generator 340, the laser beam transmitter 350 and the detector 360, respectively.

The first laser beam generator 410a, the third laser beam generator 410c and the second laser beam generator 410b generate the first laser beam, the third laser beam and the second laser beam, respectively. Preferably, the third laser beam may include a laser beam that is constant and continuous or a laser beam varying according to time.

The first cable 230a provides a power from a power supply 450 to the electromagnetic wave generator 340.

The second cable 230b transmits an electrical signal generated by the detector 360 to the image generator 430.

For instance, the first cable 230a and the second cable 230b may include a copper cable.

The dome-shaped header 300 is attached to an end portion of the flexible tube 200, and is inserted into the human body along with the flexible tube 200.

The dome-shaped header 300 houses the visible image generator 320, the electromagnetic wave generator 340, the laser beam transmitter 350, the detector 360, the first collimation lens 330a, the third collimation lens 330c, the second collimation lens 330b, the first silicon lens 370a and the second silicon lens 370b.

The visible image generator 320 is disposed inside the dome-shaped header 300, and feeds a video signal corresponding to the portion of the human body to a display apparatus 500.

For instance, the visible image generator 320 may include a lens or a camera. Moreover, the visible image generator 320 generates the video signal corresponding to the human body such as organs to be diagnosed, and feeds the video signal to the display apparatus 500. A user may recognize a current location of the high resolution endoscope in real time through a video displayed on the display apparatus 500.

That is, the user may check the current location of the high resolution endoscope by referring the video displayed on the display apparatus 500, and may move the high resolution endoscope into a desired location for a diagnosis of a tumor 700 (see FIG. 10).

The first collimation lens 330a is interposed between the first laser beam propagator 210a and the electromagnetic wave generator 340, and the first laser beam incident upon the first laser beam propagator 110a is focused and propagated to the electromagnetic wave generator 340.

The third collimation lens 330c is interposed between the third laser beam propagator 210c and the laser beam transmitter 350, and the third laser beam incident upon the third laser beam propagator 210c is focused by the third collimation lens 330c and propagated to the laser beam transmitter 350.

The second collimation lens 330b is interposed between the second laser beam propagator 210b and the detector 360, and the second laser beam incident upon the second laser beam propagator 210b is focused and propagated to the detector 360.

For instance, each of the first collimation lens 330a, the second collimation lens 330b and the third collimation lens 330c may include optical lens.

The laser beam transmitter 350 radiates the third laser beam on the portion of the human body having the contrast agent adhered thereto. It is preferable that the third laser beam includes one of an infrared laser beam and a visible ray laser beam.

When the third laser beam is radiated on the portion of the human body having the contrast agent adhered thereto, a temperature of the moisture contained in the tumor, i.e. a temperature of the portion of the human body having the contrast agent adhered thereto rises due to a surface plasmon polariton phenomenon. Therefore, the tumor may easily be located due to an increase of the contrast by the terahertz electromagnetic wave. In addition, a cancer may be killed due to the rise of the temperature.

Preferably, the contrast agent may include one of a metal nanoparticle and a metal nanocluster including the metal nanoparticle.

The metal nanoparticle may include at least one of a Pt (Platinum), a Pd (Palladium), an Ag (Silver), a Cu (Copper) and an Au (Gold). Moreover, it is preferable that the metal nanoparticle may have at least one of shapes of a rod, an oval and a sphere.

The metal nanocluster may be manufactured via an emulsion method using the metal nanoparticle.

The electromagnetic wave generator 340 radiates the terahertz electromagnetic wave excited by the first laser beam on the portion of the human body having the contrast agent adhered thereto.

The contrast agent maximizes a reaction of the terahertz electromagnetic wave radiated on the portion of the human body, thereby increasing a resolution of the imaging.

For instance, the terahertz electromagnetic wave may be excited by the first laser beam via a photoconductive antenna or an optical rectification.

The first silicon lens 370a collimates the terahertz electromagnetic wave radiated by the electromagnetic wave generator 340.

The second silicon lens 370b collimates the terahertz electromagnetic wave reflected from the portion of the human body.

Preferably, the first silicon lens 370a and the second silicon lens 370b are disposed inside the dome-shaped header 300 or on a surface of the dome-shaped header 300.

The first silicon lens 370a collimates and radiates the terahertz electromagnetic wave radiated from the electromagnetic wave generator 340. Therefore, the first silicon lens 370a may radiate the terahertz electromagnetic wave into the desired location. In addition, the second silicon lens 370b may collimate and propagate the terahertz electromagnetic wave reflected from the portion of the human body to the detector 360.

That is, when the first silicon lens 370a is used, the terahertz electromagnetic wave is radiated accurately into the desired location. Moreover, when the second silicon lens 370b is used, the terahertz electromagnetic wave reflected in a widely manner is collected and propagated.

Specifically, the terahertz electromagnetic wave generated by the electromagnetic wave generator 340 is passed through the first silicon lens 370a to be collimated and radiated on the tumor 700 as shown in FIG. 10 such that the terahertz electromagnetic wave is locally radiated. The terahertz electromagnetic wave reflected from the tumor 700 is collimated through the second silicon lens 370b and propagated to the detector 360. The user may move the high resolution endoscope in a manner that the terahertz electromagnetic wave is radiated on an entirety of the tumor 700.

The detector 360 generates an electrical signal for the imaging based on the reflected terahertz electromagnetic wave and the second laser beam.

For instance, the detector 360 generates the electrical signal using one of a photoconductivity sampling and an electro-optic sampling.

The image generator 430 generates the high resolution image based on the electrical signal received from the detector 360 and the high resolution image generated by the image generator 430 is displayed on the display apparatus 500.

As described above, the laser beam transmitter 350 in accordance with the present invention may radiate the third laser beam that is constant and continuous or the third laser beam varying according to time.

A process of generating the image generated by the image generator 430 in accordance with present invention is identical to that of the image generator 190 in accordance with present invention. Therefore, a detailed description thereof is omitted The first cable 230a provides a power from a power supply 450 to the electromagnetic wave generator 340.

The second cable 230b transmits the electrical signal generated by the detector 360 to the image generator 430.

For instance, each of the first cable 230a and the second cable 230b may include a cooper cable.

As described above, the apparatus and the method for generating the high resolution image of the human body using the terahertz electromagnetic wave and the endoscope using the same in accordance with the present invention provides following advantages.

First, the apparatus in accordance with the present invention radiates the third laser beam on the portion of the human body having the contrast agent adhered thereto to provide the high resolution image with improved a contrast.

Second, since the apparatus in accordance with the present invention generates the image using the difference of the electrical signals generated by radiating the third laser beam varying according to time on the portion of the human body, the tumor in the human body may be accurately detected.

Third, the apparatus in accordance with the present invention provides the image of the portion of the human body having the resolution of a few micrometers (μm). Therefore, the apparatus in accordance with the present invention provides the improved resolution of at least 1000 times compared to that of a few millimeters (mm) provided by a conventional image generating apparatus.

Fourth, the cancers of digestive organs such as stomach cancer and intestine cancer, which are difficult to diagnose using the conventional medical instrument such as an endoscope or MRI equipment, may be easily diagnosed by using the terahertz electromagnetic wave sensitive to the moisture.

Fifth, when the third laser beam and the terahertz electromagnetic wave is radiated on the portion of the human body having the contrast agent adhered thereto, the tumor in the human body may killed due to the rise of the temperature thereof.

While the present invention has been particularly shown and described with reference to the preferred embodiment thereof, it will be understood by those skilled in the art that various changes in form and details may be effected therein without departing from the spirit and scope of the invention as defined by the appended claims.

What is claimed is:

1. An apparatus for generating a high resolution image of a human body, the apparatus comprising:
an electromagnetic wave generator for radiating a terahertz electromagnetic wave excited by a first laser beam on a portion of the human body having a contrast agent adhered thereto;
a laser beam transmitter for radiating a third laser beam on the portion of the human body;
a detector for generating an electric signal for an imaging based on the terahertz electromagnetic wave reflected from the portion of the human body having the third laser beam radiated thereon and a second laser beam; and
an image generator for generating the high resolution image based on the electric signal received from the detector.

2. The apparatus in accordance with claim 1, further comprising:
a first laser beam generator for generating the first laser beam;
a first laser beam propagator for propagating the first laser beam generated by the first laser beam generator to the electromagnetic wave generator;
a third laser beam generator for generating the third laser beam;
a third laser beam propagator for propagating the third laser beam generated by the third laser beam generator to the laser beam transmitter;
a second laser beam generator for generating the second laser beam; and
a second laser beam propagator for propagating the second laser beam generated by the second laser beam generator to the detector.

3. The apparatus in accordance with claim 2, wherein each of the first laser beam propagator, the third laser beam propagator and the second laser beam propagator includes an optical fiber.

4. The apparatus in accordance with claim 2, further comprising:
a first collimation lens for focusing the first laser beam, the first collimation lens being interposed between the electromagnetic wave generator and the first laser beam propagator;
a third collimation lens for focusing the third laser beam, the third collimation lens being interposed between the laser beam transmitter and the third laser beam propagator; and
a second collimation lens for focusing the second laser beam, the second collimation lens being interposed between the detector and the second laser beam propagator.

5. The apparatus in accordance with claim 1, further comprising a first silicon lens for collimating the terahertz electromagnetic wave radiated by the electromagnetic wave generator.

6. The apparatus in accordance with claim 1, further comprising a second silicon lens for collimating the terahertz electromagnetic wave reflected from the portion of the human body.

7. The apparatus in accordance with claim 1, further comprising:
a first cable for providing a power to the electromagnetic wave generator from a power supply; and
a second cable for transmitting the electric signal generated by the detector to the image generator.

8. The apparatus in accordance with claim 1, wherein the terahertz electromagnetic wave is excited by the first laser beam via one of a photoconductive antenna and an optical rectification.

9. The apparatus in accordance with claim 1, wherein the detector generates the electric signal using one of a photoconductive sampling and an electro-optic sampling.

10. The apparatus in accordance with claim 1, wherein the contrast agent includes one of a metal nanoparticle and a metal nanocluster including the metal nanoparticle.

11. The apparatus in accordance with claim 10, wherein the metal nanoparticle includes at least one of shapes of a rod, an oval and a sphere.

12. The apparatus in accordance with claim 10, wherein the metal nanoparticle includes at least one of a Pt, a Pd, an Ag, a Cu and an Au.

13. The apparatus in accordance with claim 1, wherein the third laser beam includes one of an infrared laser beam and a visible ray laser beam.

14. The apparatus in accordance with claim 1, wherein the image generator generates the high resolution image based on a difference in the electric signal varying according to a variation of the third laser beam.

15. The apparatus in accordance with claim 14, wherein the third laser beam includes a square wave laser beam.

16. A high resolution endoscope comprising:
a electromagnetic wave generator for radiating an terahertz electromagnetic wave excited by a first laser beam on a portion of a human body having a contrast agent adhered thereto;
a laser beam transmitter for radiating a third laser beam on the portion of the human body;
a detector for generating an electric signal for a imaging based on the terahertz electromagnetic wave reflected from the portion of the human body having the third laser beam radiated thereon and a second laser beam;
an image generator for generating a high resolution image based on the electric signal received from the detector;
a first laser beam propagator, a third laser beam propagator and a second laser beam propagator for propagating the first laser beam, the third laser beam and the second laser beam to the electromagnetic wave generator, the laser beam transmitter and the detector, respectively;
a flexible tube; and
a dome-shaped header attached to an end portion of the flexible tube, the dome-shaped header being inserted into the human body with the flexible tube; and
wherein the dome-shaped header houses in the electromagnetic wave generator, the laser beam transmitter and the detector, and wherein the flexible tube houses in the first laser beam propagator, the third laser beam propagator and the second laser beam propagator.

17. The endoscope in accordance with claim 16, further comprising:
a first laser beam generator for generating the first laser beam;
a third laser beam generator for generating the third laser beam; and
a second laser beam generator for generating the second laser beam.

18. The endoscope in accordance with claim 16, further comprising:
a first collimation lens for focusing the first laser beam, the first collimation lens being interposed between the electromagnetic wave generator and the first laser beam propagator;
a third collimation lens for focusing the third laser beam, the third collimation lens being interposed between the laser beam transmitter and the third laser beam propagator; and
a second collimation lens for focusing the second laser beam, the second collimation lens being interposed between the detector and the second laser beam propagator.

19. The endoscope in accordance with claim 16, further comprising a first silicon lens for collimating the terahertz electromagnetic wave radiated by electromagnetic wave generator.

20. The endoscope in accordance with claim 19, wherein the first silicon lens is disposed on a surface of or inside the dome-shaped header.

21. The endoscope in accordance with claim 16, further comprising a second silicon lens for collimating the terahertz electromagnetic wave reflected from the portion of the human body.

22. The endoscope in accordance with claim 21, wherein the second silicon lens is disposed for one of an inside of the dome-shaped header.

23. The endoscope in accordance with claim 16, further comprising:
a first cable for providing a power from a power supply to the electromagnetic wave generator; and
a second cable for transmitting the electric signal generated by the detector to the image generator.

24. The endoscope in accordance with claim 23, wherein the first cable and the second cable are disposed in the flexible tube.

25. The endoscope in accordance with claim 16, wherein the terahertz electromagnetic wave is excited by the first laser beam via one of a photoconductive antenna and an optical rectification.

26. The endoscope in accordance with claim 16, wherein the detector generates the electric signal using one of a photoconductive sampling and an electro-optic sampling.

27. The endoscope in accordance with claim 16, wherein each of the first laser beam propagator, the third laser beam propagator and second laser beam propagator includes an optical fiber.

28. The endoscope in accordance with claim 16, further comprising a visible image generator for feeding a video of the portion of the human body, the visible image generator being housed in the dome-shaped header.

29. The endoscope in accordance with claim 16, wherein the contrast agent includes one of a metal nanoparticle and a metal nanocluster including the metal nanoparticle.

30. The endoscope in accordance with claim 29, wherein the metal nanoparticle includes at least one of shapes of a rod, an oval and a sphere.

31. The endoscope in accordance with claim 29, wherein the metal nanoparticle includes at least one of a Pt, a Pd, an Ag, a Cu and an Au.

32. The endoscope in accordance with claim 16, wherein the third laser beam includes one of an infrared laser beam and a visible ray laser beam.

33. The endoscope in accordance with claim 16, wherein the image generator generates the high resolution image based on a difference in the electric signal varying according to a variation of the third laser beam.

34. The endoscope in accordance with claim 33, wherein the third laser beam includes a square wave laser beam.

35. A method for generating a high resolution image of a human body, the method comprising:
(a) coating a contrast agent on a portion of the human body;
(b) radiating a terahertz electromagnetic wave excited by a first laser beam on the portion of the human body having the contrast agent adhered thereto;
(c) radiating a third laser beam on the portion of the human body;
(d) generating an electric signal for an imaging based on the terahertz electromagnetic wave reflected from the portion of the human body having the third laser beam radiated thereon and a second laser beam; and
(e) generating an image from the electric signal.

36. The method in accordance with claim 35, wherein the step (c) comprises radiating the third laser beam varying according to time, and
wherein the step (d) comprises generating the electric signal varying according to the third laser beam.

37. The method in accordance with claim 36, wherein the step (e) comprises generating the image based on a difference in electric signal varying according to the third laser beam.

38. The method in accordance with claim 35, further comprising (f) displaying the image on a display apparatus.
